(19) United States
(12) Patent Application Publication
(10) Pub. No.: US 2009/0298912 A1
(43) Pub. Date: Dec. 3, 2009

(54) Arginase II: A Target treatment of aging heart and heart failure

(75) Inventors: Dan E. Berkowitz, Baltimore, MD (US); Joshua M. Hare, Baltimore, MD (US); Artin A. Shoukas, Baltimore, MD (US); Hunter Champion, Baltimore, MD (US); Jochen Steppan, Oftersheim (DE)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 11/988,186

(22) PCT Filed: Jun. 29, 2006
(86) PCT No.: PCT/US06/25601

§ 371 Date: May 14, 2009

Related U.S. Application Data
(60) Provisional application No. 60/696,359, filed on Jul. 1, 2005.

(51) Int. Cl. A61K 31/713; A61P 9/04; C12Q 1/37; G01N 33/573

(52) U.S. Cl. 514/44 A; 435/24; 435/7.4

(57) ABSTRACT

The instant invention provides methods and compositions for the treatment of cardiac dysfunction. Specifically, the invention provides methods and compositions for modulating Arginase II for the treatment of cardiac dysfunction.

### RELATED APPLICATIONS

This application claims the benefit of U.S. Provisional Application No. 60/696,359, filed Jul. 1, 2005, the entire contents of which is expressly incorporated herein by reference.

### GOVERNMENT SUPPORT

The following invention was supported at least in part by NIH grant R01 AG021523 and National Space Biomedical Research Grant CA00203. Accordingly, the government may have certain rights in the invention.

### BACKGROUND OF THE INVENTION

Recent evidence has clearly demonstrated the critical role of NOS isoforms in the spatial confinement of NO signaling in the heart (1-3). Specifically, in the sarcoplasmic reticulum (SR), NOS1 co-localizes with the ryanodine receptor, and activation of NOS1 positively modulates cardiac contractility. Moreover, NOS1 deficiency leads to an increase in xanthine oxidase (XO)-dependent ROS activity which dramatically depresses myocardial contractile function (4). In contrast, the NOS3 isoform coupled to the beta-3 adrenergic receptor (AR), inhibits L-type Ca²⁺ channels and thus inhibits beta-AR mediated increases in myocardial contractility (5).

NO signaling may be mediated by soluble guanylyl cyclase (sGC) dependent increase in cGMP (6), or by cGMP-independent nitrosylation of a broad spectrum of effector proteins (7). An emerging body of evidence indicates that the balance between NO and O₂— regulates the nitroso-redox balance, thus, determining the nitrosylation of proteins and their resultant physiologic or pathophysiologic effects (8).

Although the activity and abundance of enzymes important in the regulation/dysregulation of the NO/redox balance in physiological and pathophysiological conditions (eg, heart failure) are currently being characterized (9), the mechanisms that regulate the pivotal NOS enzyme substrate, L-arginine, remain poorly understood. Accordingly, understanding the role of Arginase in the regulation of L-arginine would help to understand the molecular mechanisms of regulating NOS activity.

However, the role of arginase in modulating NOS activity in the heart is unknown. Accordingly, the need exists to determine the role of arginase in the modulation of NOS activity so as to better understand the molecular events that lead to myocardial dysfunction and potentially identify new targets for therapeutic treatment.

### SUMMARY OF THE INVENTION

The instant invention is based, at least in part, on the discovery that Arginase II is expressed in the heart and is located in myocyte mitochondria where it regulates NO dependent basal myocardial contractility in an NOS1 dependent manner. Furthermore, ArgII deficient mice are protected from developing heart failure.

Accordingly, in one aspect, the instant invention provides methods of treating or preventing cardiac dysfunction in a subject by administering to the subject an effective amount of a compound that inhibits the expression or activity of Arginase II, thereby treating or preventing cardiac dysfunction in a subject. In one embodiment, the cardiac dysfunction is age related cardiac dysfunction.

In another aspect, the instant invention provides methods of treating or preventing heart failure in a subject by administering to the subject an effective amount of a compound that inhibits the expression or activity of Arginase II, thereby treating or preventing heart failure in a subject.

In another aspect, the instant invention provides methods of treating or preventing vascular stiffness in a subject by administering to the subject an effective amount of a compound that inhibits the expression or activity of Arginase II, thereby treating or preventing vascular stiffness in a subject.

In another aspect, the instant invention provides methods of treating or preventing myocardial dysfunction in a subject by modulating the activity of Nitric Oxide Synthase 1 (NOS 1) by contacting an Arginase II polypeptide, or a cell expressing an Arginase II polypeptide, with a compound that inhibits the expression or activity of Arginase II, thereby modulating the activity of NOS and treating or preventing myocardial dysfunction in a subject.

In one embodiment, the compound inhibits the expression of Arginase II, e.g., by decreasing the transcription or translation of Arginase II. In a specific embodiment, the compound decreases the translation of Arginase II. In one embodiment, the compound is a nucleic acid molecule, e.g., an antisense RNA molecule, a siRNA molecule or a shRNA molecule. In a specific embodiment, the nucleic acid molecule is an siRNA molecule comprising the sequence set forth as SEQ ID NO:3.

In another embodiment, the compound inhibits the activity of Arginase II. In exemplary embodiments, the compound is a small molecule, peptide, polypeptide, or nucleic acid molecule. In a specific embodiment, the compound is a small molecule, e.g., nor-NOHA, BEC, DFMO and ABH.

In another aspect, the instant invention provides methods of determining if a subject is at risk of developing heart failure or cardiac dysfunction by obtaining a biological sample from the subject and determining the level of Arginase II in the sample, wherein an elevated level of Arginase II in the sample as compared to a control is indicative that the subject is at risk of developing heart failure or cardiac dysfunction or has undergone a myocardial infarction.

In a related embodiment, the cardiac dysfunction is age related cardiac dysfunction. In another related embodiment, the biological sample comprises cardiac myocytes. In another related embodiment, the level of Arginase II is determined by cellular imaging using a detectable antibody, e.g., an antibody specific for Arginase II.

In another aspect, the instant invention provides methods for treating or preventing age related cardiac dysfunction by modulating the activity of Arginase II comprising contacting the polypeptide or a cell expressing the polypeptide with a compound which binds to Arginase II in a sufficient concentration to modulate the activity of the to Arginase II.

In another aspect, the instant invention provides methods for identifying a compound which modulates the activity of Arginase II by contacting Arginase II, or a cell expressing Arginase II with a test compound and determining whether the test compound binds to Arginase II. In a related embodiment, the modulation of Arginase II is detected by detecting a change in the rate of Arginase II enzyme activity. In another related embodiment, the method is for the identification of a compound for the treatment or prevention of cardiac dysfunction, age related cardiac dysfunction, heart failure, decreasing vascular stiffness, decreasing oxidant stress or increasing myocardial contractility.

In another aspect, the instant invention provides methods for identifying a compound which treats or prevents cardiac dysfunction, age related cardiac dysfunction, or heart failure by modulating the activity of Arginase II comprising, contacting Arginase II with a test compound and determining the effect of the test compound on the activity of the Arginase II to thereby identify a compound which modulates the activity Arginase II and treats or prevents myocardial dysfunction.

In another aspect, the invention provides compounds for the treatment of cardiac dysfunction or heart failure identified by the method described herein. In another embodiment, the invention provides pharmaceutical compositions comprising the compounds identified by the methods described herein. In another embodiment, the invention provides compound kits comprising the pharmaceutical composition or compounds described herein and instructions for use. In specific embodiments, the kits are for the treatment of myocardial dysfunction or heart failure.

In another aspect, the invention provides kits for the diagnosis of myocardial dysfunction or heart failure comprising an antibody specific for Arginase II, and instructions for use.

### DESCRIPTION OF THE DRAWINGS

FIGS. 1A-B depict Arginase expression and activity in rat heart and myocytes. a) (i) Expression of Arg isoforms in both rat heart (H) and isolated myocyte (M) homogenates by Immunoblotting. While Arg II is confined exclusively to cardiac myocytes, Arg I and II is demonstrated in whole heart homogenates. Rat liver (L) homogenate is a positive control for Arg I and rat kidney (K) is a positive control for Arg II (ii) Immunocytochemistry demonstrating Arg II but not Arg I in isolated rat myocytes. Isolated myocytes were fixed and immunofluorescence detected with ArgII, and cy5-conjugated Anti-rabbit Abs. iii) RT-PCR confirming the mRNA expression of Arg I and II in whole heart but Arg II alone in isolated myocytes b) Arginase activity is present in both whole rat heart (n=4) and isolated rat myocytes (n=3). Although arginase activity was significantly higher in the heart than in isolated myocytes, the activity was inhibited in the presence of the specific arginase inhibitor, BEC, in a dose-dependent manner (*p<0.001 vs control).

FIGS. 2A-B depict the interaction of Arginase and NOS. a) In order to determine if there exists a molecular interaction between Arg II and NOS isoforms, cardiac myocyte lysates were immunoprecipitated with NOS1 or NOS3 Abs and immunoblotted with an Arg II Ab. In addition myocyte lysates were immunoprecipitated with Arg II Ab and immunoblotted with NOS1 and NOS3 Abs. b) Inhibition of both heart and cardiac myocyte arginase resulted in a significant (˜2 fold) increase in heart and myocyte NO production (*p<0.001). Addition of exogenous L-Arginine (0.1 mM) had no effect on myocyte NO production.

FIGS. 3A-D depict subcellular localization of arginase II in cardiac myocytes. a) Western blot of VDAC, COX IV, Arg II, and SERCA in mitochondrial (M), sarcoplasmic reticulum (SR), and cytoplasmic (C) fractions prepared from isolated cardiac myocytes. Arg II is localized predominately in the mitochondrial fraction, with some signal in the SR fraction and very little in the cytoplasmic fraction (positive control LDH). The detection of Arg II and the mitochondrial proteins VDAC and COX IV in SR fraction is suggestive of the tight association between the mitochondrial and SR compartments. This is further evidenced by the presence of SERCA in the mitochondrial fraction as well as the SR, highlighting the inability to completely separate these two fractions with our current fractionation methods. b) Western blot of co-immunoprecipitated proteins from rat myocyte lysates using anti-Arg II and anti-NOS1 antibodies. The left lane is the negative control (Arg II−/NOS1−), while the center and right lanes show proteins immunoprecipitated with NOS1 (Arg II−/NOS1+) and Arg II (Arg II+/NOS1−), respectively. Immunoprecipitation of COX IV with Arg II, as shown in the right lane, suggests mitochondrial localization of Arg II. Immunoprecipitation of Arg II and COX IV with NOS1 and NOS1 with Arg II further implies a specific molecular interaction and/or closely adjacent subcellular localization of Arg II in mitochondria and NOS1 in the SR. Immuno-electron microscopy was used to visualize Arg II using antibody-conjugated 6-nm gold beads in rat heart histological sections. c) Transmission electron micrograph at 30,000× magnification shows a nucleus (N), z-line of a myofibril (Z), and mitochondria (M) adjacent to a myofibril. The highlighted area in the center of the image is magnified in the inset at 120,000× showing a cluster of gold beads labeling Arg II (white arrow) within a mitochondrion. d) A myocyte mitochondrion (M) at 120,000× enclosing several clusters of Arg II (white arrows) primarily located at the periphery, consistent with close spatial association with the SR.

FIGS. 4A-B depict the effect of Arginase inhibition on basal myocardial contractility. a) Isolated rat cardiac myocytes were perfused with tyrodes solution with or without BEC 10⁻⁵M alone or in combination with L-NAME (10⁻⁴M). BEC increased contractility (2.1±0.14) as measured by fold change in sarcomere shortening (n=8 cells, 3 hearts *p<0.001). This response was completely inhibited with the non-specific NOS inhibitor, L-NAME (10⁻⁴ M) (#p<0.001). b) Nor-NOHA, doses-dependently increased contractility (sarcomere shortening) (1.9±0.45 fold increase, *p<0.05) the effect of which was specifically inhibited in the presence of L-NAME.

FIGS. 5A-B depict the effect of arginase inhibition on myocardial contractility is NOS1 isoform specific. a) BEC dose dependently increased SS in isolated rat myocytes (n=7 from 3 hearts, *p<0.01). This effect was inhibited by the NOS1 specific inhibitor SMTC. b) Isolated myocytes from WT, NOS1 and NOS3 mice were perfused with tyrodes solution containing increasing doses of BEC. BEC dose dependently increased SS in both WT and NOS3 deficient mice but had no effect on contractility in NOS1 deficient mice (n=11 from 3 hearts, p=n.s. from baseline or *p<0.001 vs. WT and NOS3).

FIG. 6 is a schematic demonstrating the proposed mechanism by which mitochondrial Arg II regulates NOS1-dependent myocardial contractility

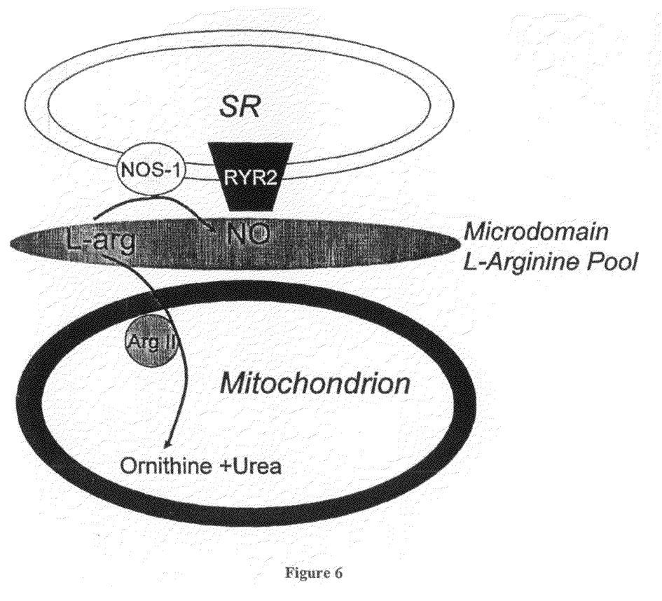

FIG. 7 depicts the results of experiments with WT and ApoE knockout mice before and after normal or high cholesterol and placebo or BEC treatment.

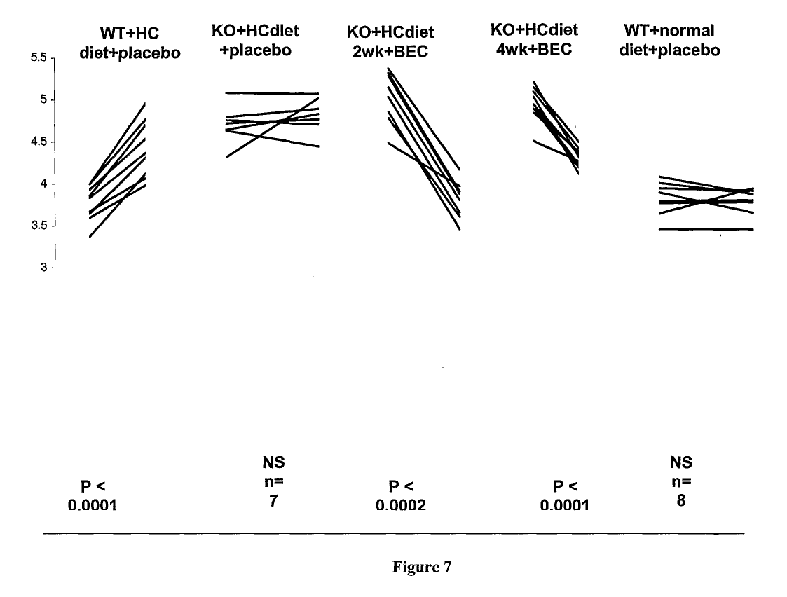

FIG. 8 depicts the results of experiments showing the ROS as determined by luminol activity.

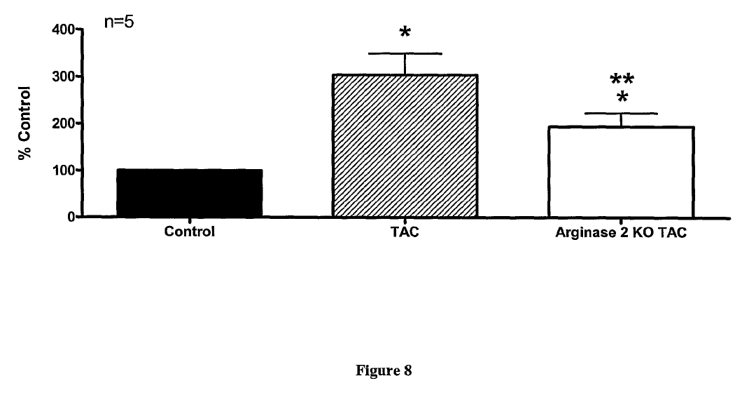

### DETAILED DESCRIPTION OF THE INVENTION

The instant invention is based, at least in part, on the discovery that arginase II is expressed in cardiac myocytes and that it regulate NOS. Moreover, NOS is known to be involved in the regulation of myocardial contractility. In addition, mice deficient in Arg II are protected from the development of heart failure. Accordingly, the instant invention provides methods and compositions to treat or prevent disorders associated with myocardial contractility.

The instant invention is directed to methods and compositions for treating conditions related to myocardial contractility. Specifically, the invention is directed to methods and compositions for the treatment of cardiac dysfunction, myocardial hypertrophy and remodeling, age related cardiac dysfunction, heart failure, decreasing vascular stiffness, decreasing oxidant stress and methods for increasing myocardial contractility by modulating the activity of Arginase II.

Accordingly, in one aspect, the invention provides a method (also referred to herein as a “screening assay”) for identifying modulators, i.e., candidate or test compounds or agents (e.g., peptides, peptidomimetics, small molecules or other drugs) which bind to Arginase II proteins or have a inhibitory effect on, for example, the expression, activity or the amount of Arginase II. The compounds tested as modulators of Arginase II can be any small organic molecule, or a biological entity, such as a protein, e.g., an antibody or peptide, a sugar, a nucleic acid, e.g., an antisense oligonucleotide, RNAi, or a ribozyme, or a lipid. Typically, test compounds will be small organic molecules, peptides, lipids, and lipid analogs.

In one embodiment, the invention provides assays for screening candidate or test compounds which are substrates of an Arginase II protein or polypeptide or biologically active portion thereof. In another embodiment, the invention provides assays for screening candidate or test compounds which bind to or modulate the activity of an Arginase II protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses an Arginase II protein or biologically active portion thereof is contacted with a test compound and the ability of the test compound to modulate Arginase II activity is determined. Determining the ability of the test compound to modulate Arginase II activity can be accomplished by monitoring, for example, intracellular calcium, IP3, or diacylglycerol concentration, phosphorylation profile of intracellular proteins, cell proliferation and/or migration, or the activity of an Arginase II-regulated transcription factor. The cell, for example, can be of mammalian origin, e.g., a myocyte.

The ability of the test compound to modulate Arginase II binding to a substrate or to bind to Arginase II can also be determined. Determining the ability of the test compound to modulate Arginase II binding to a substrate can be accomplished, for example, by coupling the Arginase II substrate with a radioisotope or enzymatic label such that binding of the Arginase II substrate to Arginase II can be determined by detecting the labeled Arginase II substrate in a complex. Alternatively, Arginase II could be coupled with a radioisotope or enzymatic label to monitor the ability of a test compound to modulate Arginase II binding to a Arginase II substrate in a complex. Determining the ability of the test compound to bind Arginase II can be accomplished, for example, by coupling the compound with a radioisotope or enzymatic label such that binding of the compound to Arginase II can be determined by detecting the labeled Arginase II compound in a complex. For example, compounds (e.g., Arginase II substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., an Arginase II substrate) to interact with Arginase II without the labeling of any of the interactants. For example, a microphysiometer can be used to detect the interaction of a compound with Arginase II without the labeling of either the compound or the Arginase II. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and Arginase II.

In another embodiment, an assay is a cell-based assay comprising contacting a cell expressing an Arginase II target molecule (e.g., an Arginase II substrate) with a test compound and determining the ability of the test compound to modulate (e.g., stimulate or inhibit) the activity of the Arginase II target molecule. Determining the ability of the test compound to modulate the activity of an Arginase II target molecule can be accomplished, for example, by determining the ability of the Arginase II protein to bind to or interact with the Arginase II target molecule.

Determining the ability of the Arginase II protein or a biologically active fragment thereof, to bind to or interact with an Arginase II target molecule can be accomplished by one of the methods described above for determining direct binding. In a preferred embodiment, determining the ability of the Arginase II protein to bind to or interact with an Arginase II target molecule can be accomplished by determining the activity of the target molecule. For example, the activity of the target molecule can be determined by detecting induction of a cellular second messenger of the target (i.e., intracellular Ca²⁺, diacylglycerol, IP₃, and the like), detecting catalytic/enzymatic activity of the target an appropriate substrate, detecting the induction of a reporter gene (comprising a target-responsive regulatory element operatively linked to a nucleic acid encoding a detectable marker, e.g., luciferase), or detecting a target-regulated cellular response.

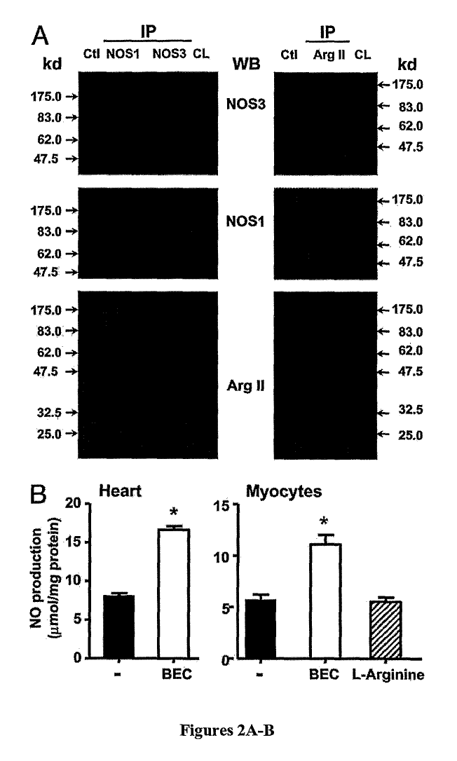

In yet another embodiment, an assay of the present invention is a cell-free assay in which an Arginase II protein or biologically active portion thereof is contacted with a test compound and the ability of the test compound to bind to the Arginase II protein or biologically active portion thereof is determined. Preferred biologically active portions of the Arginase II proteins to be used in assays of the present invention include fragments which participate in interactions with non-Arginase II molecules, e.g., fragments with high surface probability scores (see, for example, FIGS. 2 and 13). Binding of the test compound to the Arginase II protein can be determined either directly or indirectly as described above. In a preferred embodiment, the assay includes contacting the Arginase II protein or biologically active portion thereof with a known compound which binds Arginase II to form an assay mixture, contacting the assay mixture with a test compound, and determining the ability of the test compound to interact with an Arginase II protein, wherein determining the ability of the test compound to interact with an Arginase II protein comprises determining the ability of the test compound to preferentially bind to Arginase II or biologically active portion thereof as compared to the known compound.

In another embodiment, the assay is a cell-free assay in which an Arginase II protein or biologically active portion thereof is contacted with a test compound and the ability of the test compound to modulate (e.g., stimulate or inhibit) the activity of the Arginase II protein or biologically active portion thereof is determined. Determining the ability of the test compound to modulate the activity of an Arginase II protein can be accomplished, for example, by determining the ability of the Arginase II protein to bind to an Arginase II target molecule by one of the methods described above for determining direct binding. Determining the ability of the Arginase II protein to bind to an Arginase II target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of an Arginase II protein can be accomplished by determining the ability of the Arginase II protein to further modulate the activity of a downstream effector of an Arginase II target molecule. For example, the activity of the effector molecule on an appropriate target can be determined or the binding of the effector to an appropriate target can be determined as previously described.

In yet another embodiment, the cell-free assay involves contacting an Arginase II protein or biologically active portion thereof with a known compound which binds the Arginase II protein to form an assay mixture, contacting the assay mixture with a test compound, and determining the ability of the test compound to interact with the Arginase II protein, wherein determining the ability of the test compound to interact with the Arginase II protein comprises determining the ability of the Arginase II protein to preferentially bind to or modulate the activity of an Arginase II target molecule.

In more than one embodiment of the above assay methods of the present invention, it may be desirable to immobilize either Arginase II or its target molecule to facilitate separation of complexed from uncomplexed forms of one or both of the proteins, as well as to accommodate automation of the assay. Binding of a test compound to an Arginase II protein, or interaction of an Arginase II protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/Arginase II fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or Arginase II protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of Arginase II binding or activity determined using standard techniques.

Other techniques for immobilizing proteins on matrices can also be used in the screening assays of the invention. For example, either an Arginase II protein or an Arginase II target molecule can be immobilized utilizing conjugation of biotin and streptavidin. Biotinylated Arginase II protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with Arginase II protein or target molecules but which do not interfere with binding of the Arginase II protein to its target molecule can be derivatized to the wells of the plate, and unbound target or Arginase II protein trapped in the wells by antibody conjugation. Methods for detecting such complexes, in addition to those described above for the GST-immobilized complexes, include immunodetection of complexes using antibodies reactive with the Arginase II protein or target molecule, as well as enzyme-linked assays which rely on detecting an enzymatic activity associated with the Arginase II protein or target molecule.

In another embodiment, modulators of Arginase II expression are identified in a method wherein a cell is contacted with a candidate compound and the expression of Arginase II mRNA or protein in the cell is determined. The level of expression of Arginase II mRNA or protein in the presence of the candidate compound is compared to the level of expression of Arginase II mRNA or protein in the absence of the candidate compound. The candidate compound can then be identified as a modulator of Arginase II expression based on this comparison. For example, when expression of Arginase II mRNA or protein is greater (statistically significantly greater) in the presence of the candidate compound than in its absence, the candidate compound is identified as a stimulator of Arginase II mRNA or protein expression. Alternatively, when expression of Arginase II mRNA or protein is less (statistically significantly less) in the presence of the candidate compound than in its absence, the candidate compound is identified as an inhibitor of Arginase II mRNA or protein expression. The level of Arginase II mRNA or protein expression in the cells can be determined by methods described herein for detecting Arginase II mRNA or protein.

In yet another aspect of the invention, the Arginase II proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with Arginase II (“Arginase II-binding proteins” or “Arginase II-bp”) and are involved in Arginase II activity.

The two-hybrid system is based on the modular nature of most transcription factors, which consist of separable DNA-binding and activation domains. Briefly, the assay utilizes two different DNA constructs. In one construct, the gene that codes for an Arginase II protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an Arginase II-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the Arginase II protein.

Moreover, the ability of a test compound to inhibit the release of Arginase II from microtubules can be monitored as described in the examples. For example, an antibody specific for Arginase II can be used to visualize the location of Arginase II within a cell. Additionally, a second antibody specific for the microtubules can be visualized within the cell and the skilled artisan can determine if the Arginase II is bound to the microtubules. The ability of a compound to modulate the release of Arginase II from microtubules can therefore be monitored visually as described herein.

In another aspect, the invention pertains to a combination of two or more of the assays described herein. For example, a modulating agent can be identified using a cell-based or a cell free assay, and the ability of the agent to modulate the activity of an Arginase II protein can be confirmed in vivo, e.g., in an animal such as an animal model for atherogenesis.

This invention further pertains to novel agents identified by the above-described screening assays. Accordingly, it is within the scope of this invention to further use an agent identified as described herein in an appropriate animal model. For example, an agent identified as described herein (e.g., an Arginase II modulating agent, an antisense Arginase II nucleic acid molecule, an Arginase II-specific antibody, or an Arginase II-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

The present invention encompasses agents which modulate expression, activity or amount of Arginase II. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

The modulators of Arginase II of the invention may also be RNAi molecules. As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence or knockdown the expression of target genes, e.g., arginase II.

“RNAi molecule” or an “siRNA” refers to a nucleic acid that forms a double stranded RNA, which double stranded RNA has the ability to reduce or inhibit expression of a gene or target gene when the siRNA expressed in the same cell as the gene or target gene. “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 nucleotides in length, e.g., 20, 21, 22, 23, 24, 25, 26, 27, 28, 29, or 30 nucleotides in length.

The modulators of Arginase II of the invention may also be antibodies. “Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1}, by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3rd ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and coworkers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to Arginase II, polymorphic variants, alleles, orthologs, and conservatively modified variants, or splice variants, or portions thereof, can be selected to obtain only those polyclonal antibodies that are specifically immunoreactive with Arginase II and not with other proteins. This selection may be achieved by subtracting out antibodies that cross-react with other molecules. A variety of immunoassay formats may be used to select antibodies specifically immunoreactive with a particular protein.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The pharmaceutical compositions can be included in a kit, e.g., a container, pack, or dispenser, together with instructions for administration.

### Pharmaceutical Compositions

The modulators of Arginase II expression or activity described herein can be incorporated into pharmaceutical compositions suitable for administration. Such compositions typically comprise a small molecule, nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g. for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a compound (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments.

### Methods of Treatment

The present invention provides for both prophylactic and therapeutic methods of treating a subject at risk of (or susceptible to) a disorder or having a disorder associated with aberrant or unwanted Arginase II expression, regulation or activity, e.g. heart failure. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.)

Prophylactic Methods

In one aspect, the invention provides a method for preventing in a subject, a disease or condition associated with an aberrant or unwanted Arginase II expression or activity, e.g., heart failure, by administering to the subject an agent which modulates Arginase II expression or Arginase II activity. Subjects at risk for a disease which is caused or contributed to by aberrant or unwanted Arginase II expression or activity can be identified by, for example, any or a combination of diagnostic or prognostic assays as described herein. Administration of a prophylactic agent can occur prior to the manifestation of symptoms characteristic of the disease or disorder, such that a disease or disorder is prevented or, alternatively, delayed in its progression. The appropriate agent can be determined based on screening assays described herein.

Therapeutic Methods

Another aspect of the invention pertains to methods of modulating the expression of activity of Arginase II for therapeutic purposes. The methods and composition of the instant invention are useful in the treatment of, for example, heart conditions in which myocardial NO signaling is altered. Accordingly, in an exemplary embodiment, the modulatory methods of the invention involve contacting a cell with an agent that modulates Arginase II protein activity or the transcription or translation of Arginase II nucleic acid in a cell. An agent that modulates Arginase II protein activity can be an agent as described herein, such as a nucleic acid or a protein, an Arginase II antibody, an Arginase II agonist or antagonist, a peptidomimetic of an Arginase II agonist or antagonist, or other small molecule. In one embodiment, the agent inhibits the activity of Arginase II. Examples of such inhibitory agents include antisense Arginase II nucleic acid molecules, anti-Arginase II antibodies, and Arginase II inhibitors. Exemplary Arginase II inhibitors that are known in the art include, e.g., N-hydroxy-nor-L-arginine (Nor-NOHA) and S-(2-boronoethyl)-L-cysteine (BEC). These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of an Arginase II protein or nucleic acid molecule. In one embodiment, the method involves administering an agent (e.g., an agent identified by a screening assay described herein), or combination of agents that modulates (e.g., upregulates or downregulates) Arginase II expression or activity. In another embodiment, the method involves administering an Arginase II inhibitory molecule, e.g., a small molecule, protein or nucleic acid molecule, as therapy to compensate for reduced, aberrant, or unwanted Arginase II expression or activity.

In particular embodiments, the therapeutic methods of the invention are useful for treating myocardial dysfunction in which NO signaling is disrupted.

In another embodiment, the instant invention provides stents, e.g., vascular and coronary stents, comprising the Arg II modulators described herein.

Diagnostic Methods

The instant invention provides diagnostic methods for determining if a subject has, or is as risk of developing, heart failure, or other myocardial dysfunction, e.g., myocardial dysfunction in which NO signaling is disrupted. In one embodiment, the levels Arginase II are determined in a sample obtained from a subject and the levels are compared to the levels in a control sample, or to a normal level, wherein in increase in the amount of Arginase II is characteristic of a subject having, or at risk of developing myocardial dysfunction in which NO signaling is disrupted.

In another embodiment, the invention provides a method for characterizing a subjects risk profile of developing a future myocardial dysfunction in which NO signaling is disrupted comprising obtaining a level Arginase II in a sample obtained from the subject and comparing the level of Arginase II to a predetermined Arginase II value to establish a risk value, and characterizing the subject's risk profile of developing a future myocardial dysfunction based upon a combination of the risk value associated with increased levels of Arginase II.

In a related embodiment, the instant invention also provides kits for the diagnosis of myocardial dysfunction. The kit comprises a reagent that specifically detects Arginase II and instructions for use. In a specific example the kit comprises a antibody specific for Arginase II and instructions for use.

### EXAMPLES

It should be appreciated that the invention should not be construed to be limited to the examples that are now described; rather, the invention should be construed to include any and all applications provided herein and all equivalent variations within the skill of the ordinary artisan.

Methods

Reagents: S-(2-boronoethyl)-L-cysteine (BEC) and N-hydroxy-nor-L-arginine (NorNOHA) were obtained from Calbiochem. The rest of the chemical reagents were obtained from Sigma.

Animals: Mice (8 to 10 weeks old) homozygous for targeted disruption of the NOS1 gene (NOS1^{-/-}, n=3), the NOS3 gene (NOS3^{-/-}, n=3), and wild-type control mice (WT, C57BL6J, n=3) were purchased from Jackson Laboratories. All rats (Wistar, 11 to 14 weeks old) were purchased from Harlan Laboratory. All protocols conformed to the current National Institutes of Health and American Physiological Society Guidelines for the Use and Care of Laboratory Animals.

Western Blot and Co-Immunoprecipitation: Heart tissue and isolated cardiac myocyte protein of lysates were immunoprecipitated with or without 2 μg of Arginase II, NOS3 (BD biosciences) or NOS1 (Santa Cruz Biotech) antibody (rabbit, Santa Cruz Biotech. Inc.) overnight at 4° C. After incubation with protein A/G agarose for 4 h at 4° C., the beads were washed with lysis buffer for 3 times. Agarose beads were subjected to SDS-PAGE sample buffer and resolved on a 10% SDS-PAGE and immunoblotted with a monoclonal antibody against NOS1, monoclonal NOS3, or polyclonal Arg II (overnight, 4° C., 1:1,000, Santa Cruz Biotech, Inc). Antibody was detected with enhanced chemiluminescence system (Amersham).

RT-PCR: Total RNA from rat heart and isolated myocytes was prepared by homogenization in the presence of Trizol Reagent (Gibco) and RT PCR performed with specific Arg I and II primers as previously described (52).

Immunofluorescence: Isolated myocytes from rats were fixed with acetone:ethanol (3:7, V/V) solution at 4° C. for overnight and permeabilized with 3% paraformaldehyde and 0.5% Triton X-100 in PBS, rinsed with PBS and incubated with monoclonal antibody against Arginase I (BD Bioscience) or polyclonal antibody against Arg II (Santa Cruz Biotechnol. Inc) and then with DAPI conjugated anti-mouse IgG or Cy5 conjugated-anti-rabbit IgG antibody. Washed myocytes were examined with a confocal fluorescence microscope (Zeiss LSM 410).

Isolation of SR and Mitochondria Preparation: We prepared SR fractions according to the method previously described by Khan et al (4). Purified SR fractions were resolved electrophoretically and probed with anti-arginase II (Santa cruz Biotech), anti-SR Ca²⁺ ATPase (anti-SERCA2a, Affinity Bioreagents, Golden, Colo.), and anti-NOS1 (BD Transduction Laboratories, Lexington, Ky.) antibodies.

Mitochondria were prepared using the mitochondria isolation kit for tissue (Pierce Co.) following the protocol for hard tissue.

Immuno-Electron Microscopy: Immunoelectron microscopy was performed by standard procedures. Briefly, adult Wistar rats were deeply anesthetized, hearts were removed and retrogradely perfused with 4% PFA-0.05% glutaraldehyde in PBS and postfixed overnight at 4° C. 100-μm-thick vibratome sections were cut, and collected in PBS followed by incubation in the primary antibodies (rabbit anti-arginase-II diluted 1:50) for 24 h at 4° C. After washing the secondary antibody labeled with 6 nm gold particles were applied, and the tissue sections were examined with an electron microscope.

Arginase Activity: Rat hearts and myocytes were homogenized in lysis buffer (50 mM Tris-HCl, pH 7.5, 0.1 mM EDTA and protease inhibitor) and centrifuged for 30 min at 14,000 g at 4° C. for an arginase activity assay as described previously (20).

NOS Activity and NO Production: NO production was evaluated by measuring nitrite levels (Calbiochem) following pre-incubation of heart and myocytes with BEC (10 μmol/L) in PBS (pH 7.4) as previously described (52).

Measurement of Contractility in Isolated Rat and Mouse Myocytes: Both rat and mouse myocytes were isolated by enzymatic digestion as previously described (2, 3). Myocytes were transferred to a lucite chamber on the stage of an inverted microscope (Nikon TE 200), continuously superfused with Tyrodes containing 1.0 mM Ca²⁺, and stimulated at 1 Hz. Sarcomere length was recorded with an IonOptix intensified charged coupled device camera (iCCD) camera. Change in average sarcomere length was determined by fast Fourier transform of the Z-line density trace to the frequency domain (IonOptix, Milton, Mass.) as previously described (2, 3).

Measurement of ROS: ROS generation was examined by several independent methods. Superoxide production in LV tissue homogenates was determined by luminol-enhanced chemiluminescence (EMD Biosciences). Flash-frozen myocardium was homogenized in iced PBS buffer and centrifuged, and the precipitate was resuspended in

Heart Failure Model: Pressure overload was produced by TAC as previously described.

Data Analysis and Statistics: All data are presented as mean±SEM, with N being indicated for each experimental protocol. For dose responses, data was fitted using the software program Prism 4 (Graphpad) and Eₘₐₓ and EC₅₀ calculated. Statistical analysis was performed using one-way analysis of variance with post test or unpaired Student t test where appropriate.

Results

Arginase Expression and Activity in Cardiac Myocytes:

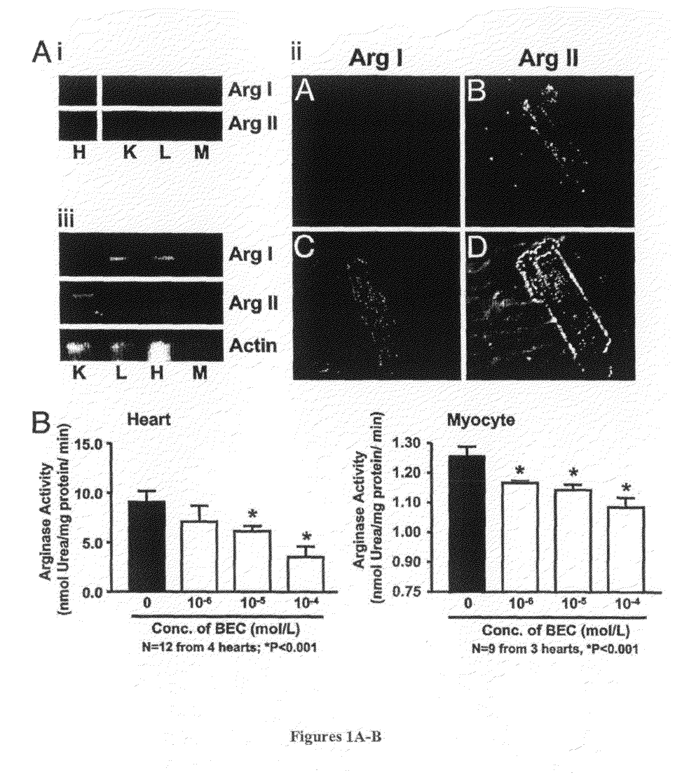

To determine whether Arg was expressed in heart tissue and isolated myocytes the following experiments were preformed. Western Blots were performed on proteins extracted from freshly isolated cardiac myocytes following collagenase digestion, as well as in homogenates of whole rat heart (rat liver was used as a control for Arg I and kidney as a control for Arg II). FIG. 1a demonstrates the expression of Arg II in isolated myocytes. While Arg II is expressed exclusively in the cardiac myocytes, both Arg I and II are found in whole heart homogenates. This most likely reflects the arginase which is present in cell types other than myocytes such as endothelial cells that have been shown to express Arg I (10, 20). Consistent with the Western blot data, immunostaining demonstrated Arg II but not Arg I in isolated myocytes (FIG. 1a). In order to confirm the above findings RT-PCR was performed using mRNA derived from isolated myocytes and whole heart (FIG. 1a). Supporting the protein expression data, Arg II mRNA is expressed solely in the isolated myocytes while both isoforms are expressed in the whole heart. Arginase activity in the heart and isolated myocytes was investigated. Arginase activity was detected in cardiac tissue and was inhibitable by the specific arginase inhibitor, BEC, in a dose-dependent manner (FIG. 1b). Because arginase is expressed and exhibits activity in non-myocyte cells in the heart, eg, endothelial cells, arginase activity in isolated cardiac myocytes was measured. Although Arg activity is lower in myocytes compared to heart tissue, this activity is inhabitable by BEC in a dose-dependent fashion (FIG. 1b).

Interaction of Arginase and NOS:

The following experiments were performed to determine whether there exists a molecular interaction between arginase II and NOS isoforms. Cardiac myocyte protein lysates were co-immunoprecipitated with NOS1 and NOS3 specific antibodies (Abs) and Western blots preformed with Arg II antibodies. In addition lysates were immunoprecipitated with Arg II antibody and WB performed with NOS1 or NOS 3 Abs. As demonstrated in FIG. 2a, Arg II was detected in lysates immunoprecipitated with NOS1 but NOS3 Abs. In addition, NOS1 but not NOS3 was detected in lysates immunoprecipitated with Arg II. This is consistent with a specific molecular interaction and/or common or closely adjacent subcellular localization between NOS1 and Arg II.

Next, it was determined whether arginase could reciprocally regulate NOS activity. NO production was measured in both heart lysates as well as lysates from isolated cardiac myocytes. BEC-induced inhibition of arginase significantly increased NO production in both the heart (16.7±1 vs. 8.07, μmol/mg protein, n=6, p<0.001) and isolated myocyte lysates (11.1±2.2 vs. 5.7±1.2 μmol/mg protein, n=6, p<0.001) (FIG. 2b). This is consistent with the hypothesis that arginase constrains NOS activity, most likely by limiting substrate availability. Interestingly, the addition of exogenous L-arginine (0.1 mM) alone to the assay buffer did not effect NO production by isolated myocytes. This supports the idea of specific pools of L-arginine being available to NOS isoforms, some of which may not be influenced by extracellular L-arginine (21, 22).

Subcellular Localization of Arginase in Cardiac Myocytes

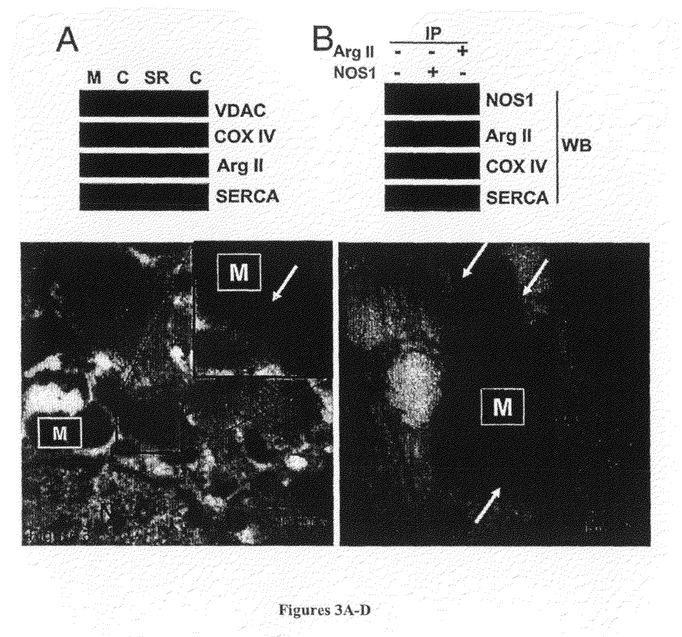

Based on the molecular association between Arg II and NOS, the subcellular localization of Arg II was investigated. NOS1 has been previously demonstrated to reside in the SR and in mitochondria (for review (23)). In the SR NOS1 is closely associated with the RYR (3, 24) where it likely regulates its nitrosylation state and thereby its capacity to release Ca²⁺ (3, 24). Given the tight association between the SR and mitochondria, an association which critically regulates coupling of cardiac excitation and oxidative energy production in the mitochondria, and given that Arg II is known to contain a putative leader sequence that targets it to the mitochondria (25, 26), experiments were designed to examine the subcellular location of Arg II within the cardiac myocyte. Both mitochondria and crude SR fractions were prepared from rat heart homogenates. As demonstrated in FIG. 3a, Arg II is detected in the mitochondrial protein fraction with very little present in the cytoplasmic fraction (positive control is LDH). SERCA is also present in proteins prepared from this mitochondrial fraction. VDAC, the voltage-dependent anion channel present only on the outer mitochondrial membrane, was used as the positive control. Because of the difficulty of isolating the mitochondria from the SR by subcellular fractionation, we attempted to determine whether Arg II was confined to the mitochondria or was present in the SR in intact cardiac myocytes. Co-immunoprecipitation of rat heart lysates with Arg II demonstrated a tight association of Arg II with the mitochondrial protein cytochrome oxidase IV (COX IV) (FIG. 3b), implying a predominantly mitochondrial localization of Arg II. In order to definitively define the spatial location of the Arg II enzyme, immuno-gold staining and electron microscopy in rat heart tissue was performed. As is seen in FIG. 3c, Arg II immuno-gold staining is confined predominantly to the mitochondria within the cardiac myocyte. Further, as shown in FIG. 3d, Arg II appears to localize primarily to the periphery of the myocyte mitochondrion, providing direct visual evidence of the Arg II enzyme within the mitochondria at locations that would facilitate close interaction with proteins in the SR membrane.

Effect of Arginase-NOS Interaction on Myocardial Contractility:

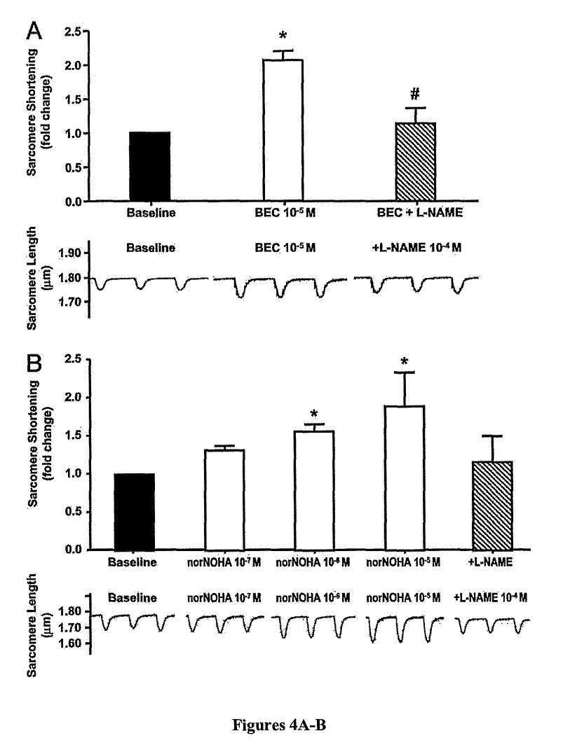

The physiologic effects of arginase on basal myocardial contractility was investigated by examining the effect of arginase inhibition on isolated myocyte sarcomere shortening (SS). Sarcomere shortening (SS) was measured in isolated myocytes in a perfusion chamber before and after the addition of the specific arginase inhibitors, BEC or Nor-NOHA (FIG. 4). Given the observation that Arg II appears to be associated with NOS1, and that NOS1 derived NO accentuates myocardial contractility, it was hypothesized that inhibition of arginase would increase basal contractility. Consistent with our hypothesis, BEC increased myocardial contractility in a dose dependent manner [LogEC₅₀; −5.8±0.9, Eₘₐₓ; 1.8±0.3 (fold increase) (FIG. 4a)]. Moreover, L-NAME (0.1 mM) completely abolished the increase in contractility observed with arginase inhibition (BEC 2.1±0.14 vs. BEC+L-NAME 1.1±0.23, p<0.001) such that the Eₘₐₓ was similar to baseline (BEC+L-NAME 1.1±0.23 vs Baseline 1.0, ns). This demonstrates that arginase inhibition exerts its effect by a NOS dependent mechanism. In addition, and consistent with our observations, incubation of cardiac myocytes with Nor-NOHA, a pharmacologically distinct specific arginase inhibitor, also caused a dose-dependent increase in basal myocardial contractility (EC₅₀ LogEC₅₀; −5.8±0.8, Eₘₐₓ; 1.98±0.23) (FIG. 4b). The EC₅₀'s for BEC and nor-NOHA are consistent with the Ki's of the inhibitors for arginase as previously published (27).

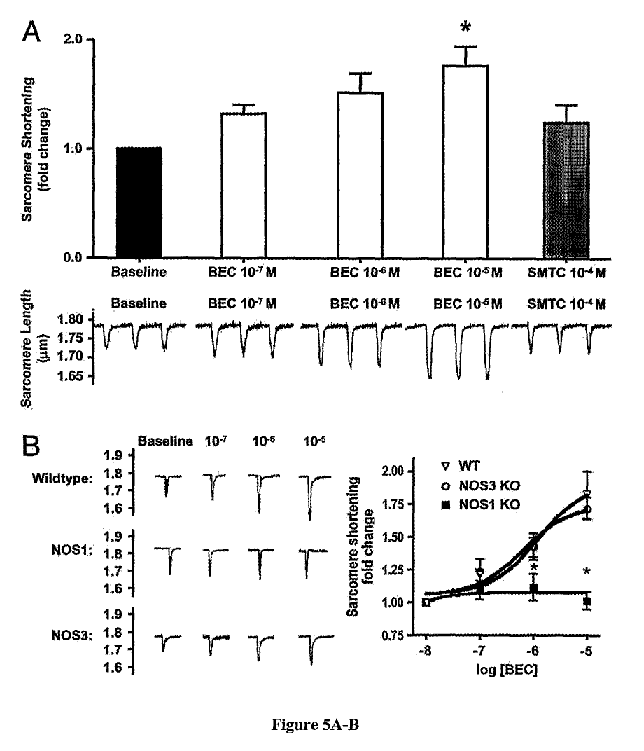

We next investigated which NOS isoform is being constrained by arginase (FIG. 5). SMTC (10 μM), a specific NOS1 inhibitor, abolished the increase in contractility observed with BEC (BEC 2.06±0.14 vs. BEC+SMTC 1.24±0.161 p<0.001) (FIG. 5). We next utilized wild type and NOS1 or NOS3 deficient mice to determine the effect of arginase inhibition on basal contractility. As illustrated in FIG. 5b, BEC caused a dose-dependent increase in basal SS in both wild type (Eₘₐₓ 1.97±0.24) and NOS3 deficient (Eₘₐₓ, 1.81±0.17) mice. In marked contrast, there was no increase in contractility, as measured by SS, in myocytes from NOS1 deficient mice (Eₘₐₓ 1.11±0.08 p<0.001 vs NOS3 and WT). While L-NAME alone resulted in a small but significant reduction in SS (0.76±0.06 fold change, n=3), L-arginine (0.1 mM) alone had no effect on myocyte contractility (1.1±0.05, n=3, ns). This is in agreement with the findings that exogenous L-arginine has no effect on myocyte NO production. Taken together, this physiologic data demonstrate that arginase constrains NOS1 activity and thereby NOS1-dependent myocardial contractility.

### ArgII KO Mice and Heart Failure

Following 3 weeks of TAC both WT and ArgII KO mice underwent hemodynamic measurements to determine the effect of TAC on cardiac function, remodeling and oxidative stress. As can be seen from Table I, there is a significant increase in cardiac mass in TAC mice.

TABLE I

| | Control | TAC | Arginase 2 KO |
|---|---|---|---|
| | N = 6 | N = 5 | N = 5 |
| Heart weight | 112 ± 5.2 | 258 ± 8.5* | 171.9 ± 21.3*† |
| (mg) | | | |
| Body weight | 26.9 ± 0.3 | 25.8 ± 2.8 | 26.2 ± 2.6 |
| (g) | | | |
| EF (%) | 65.8 ± 1.9 | 31.6 ± 4.3* | 50.5 ± 4.8*† |
| Tau (msec) | 7.0 ± 0.3 | 11.9 ± 1.3* | 8.3 ± 0.4*† |

*P < 0.05 compared to Control
†P < 0.05 compared to TAC

In addition there is a marked decrease in contractile function as measured by a decrease in ejection fraction (% EF). However, in ArgII KO mice there is a marked attenuation of the hypertrophic response to TAC. Furthermore there is a preservation of EF compared to WT TAC. While TAC induces a significant increase in oxidative stress as measured my luminol chemiluminescence, this effect was attenuated in TAC mice. Thus ArgII KO mice are protected from oxidant stress, hypertrophy and a decline in contractile function.

Discussion

The foregoing experiments have demonstrated that arginase is present predominantly in the mitochondria of cardiac myocytes where it inhibits NOS1 activity, thereby regulating NO production and ultimately basal myocardial contractility. These novel observations shed further insights into myocardial NO signaling and its spatial confinement. It appears that not only are the physiologic effects of NO defined by the specific isoform and its micro-domain within the cell, but is further regulated by the availability of substrate within that enzyme domain. These results demonstrate the complexities of the regulatory mechanisms controlling myocardial contractile function and highlight another protein that exerts a regulatory interaction with NOS1.

Spatial Confinement of No Signaling in the Heart

Although it has been recognized for over a decade that NOS isoforms are present in the heart, it is only recently that their functional role in the regulation of E-C coupling has been elucidated. It is now established that NO modulates the activity of a number of key ion channels and proteins that regulate Ca²⁺ release and thereby modulate E-C coupling. Moreover, NO can either accentuate or attenuate myocardial contractility. The foregoing experiments have demonstration that arginase interacts with NOS1 and selectively regulates its activity

Nitroso-Redox Balance/Imbalance in the Normal and Failing Heart

Nitrosylation, a highly conserved post-translational mechanism, is now recognized to regulate the function of a spectrum of proteins (8). Nitrosylation, the covalent attachment of a nitrogen monoxide group to the thiol side-chain of cysteine, is dependent on the redox milieu in that region of the protein. The ratio of superoxide versus NO production by NOS is an important determinant of the redox milieu. It is now established that both skeletal (32), and cardiac (31) ryanodine receptors are, in fact, activated by S-nitrosylation (33). The cardiac ryanodine isoform, which is s-nitrosylated under basal conditions, has been shown to co-localize with NOS1 in the SR (24, 34). NOS1 positively modulates contractility, as demonstrated by depressed force frequency and beta-adrenergic inotropic responses in NOS1 deficient mice (2, 3). Taken together, these data are consistent with the premise that NOS1 modulates the activation of ryanodine receptors, perhaps via alterations in the redox milieu and levels of ryanodine receptor nitrosylation. The foregoing results indicate that inhibition of arginase enhances basal myocardial contractility, and demonstrates that arginase modulates NOS1 and its products, superoxide and NO. Specifically, the enhanced basal contractility observed with arginase inhibition is abolished in the presence of the specific NOS1 inhibitor SMTC. Furthermore, the response to arginase inhibition is absent in NOS 1 deficient mice, but preserved in NOS3 deficient mice.

It has recently been shown that constitutive NOS isoforms contribute to the heart failure phenotype. For example, NOS3 signaling may be enhanced in heart failure. This can result from alterations in its regulatory pathways, eg, beta-3 AR signaling (39, 40) or alterations in caveolin (28). Damy et al (34) demonstrated a disruption of the spatial localization of NOS1 (translocation from SR to sarcolemma) in tissue from patients with cardiomyopathy. Moreover, NOS1 was demonstrated to be upregulated in these conditions. In the sarcolemma, NOS may inhibit contractility by modulating L-type Ca⁺⁺ channels. Since Arg is upregulated in a number of pathophysiologic states, it is interesting to speculate whether arginase upregulation may contribute to pathogenesis of heart failure.

Arginase, L-Arginine Pools and Reciprocal Regulation of NOS

Previous experiments have demonstrated that endotoxin (LPS) administration in macrophages resulted in the co-induction of the arginase isoforms Arg I and Arg II, and iNOS, leading to the hypothesis that arginase may limit sustained overproduction of NO by limiting substrate availability to iNOS (12, 26, 41, 42). Recently Arg I and Arg II expression have been demonstrated in the rat lung where they modulate cholinergic airway responses and NO activity (43). Arg I and Arg II expression has also been demonstrated in the penis (11, 16) and in A293 cells overexpressing NOS1 (44) where there exists reciprocal regulation of arginase and constitutive NOS1. Previous experiments have demonstrated (10, 20, 45, 46), that arginase isoforms are expressed constitutively in vascular endothelium and may, as in the airway, the penis, and A293 cells, modulate NOS activity by regulating L-arginine availability.

The intracellular concentration of L-arginine in endothelial cells exceeds by two to three fold its Kₘ for the NOS enzyme, indicating that L-arginine availability should not limit NOS activity or NO production. Moreover, exogenous L-arginine administration should not influence NOS activity and NO production. However, in certain conditions (diabetes, hypertension, hypercholesterolemia), the addition of extracellular L-arginine does enhance NO-dependent relaxation. Furthermore, spatial confinement of NOS1 and arginase suggests very tight control of L-arginine availability. In addition, the presence of endogenous NOS inhibitors may further exacerbate this paradox. Finally, the presence of distinct intracellular L-arginine pools may be important in determining substrate availability.

The data presented herein demonstrate that exogenous L-arginine had no effect on myocyte NO production or myocyte contractility is consistent with the idea of different L-arginine pools in cardiac myocyte specifically, but in other cell in general. This issue also gets to the heart of the arginine paradox described above. The fact that exogenous L-Arginine in our experiments has little effect on NOS activity in the myocyte demonstrates that the pool of L-Arginine which is available to NOS- may not be in regulated by the CAT transporter.

Mitochondrial Arg and SR Coupling

While myocyte subcellular fractionation and immunoblotting suggested that Arg II was predominantly found in the mitochondria, immuno-electron microscopy conclusively demonstrated that Arg II is almost exclusively confined to the mitochondria. This is in agreement with the findings of others who demonstrate Arg II confined to the mitochondria in other cell types (49, 50) and is consistent with the putative amino terminal mitochondrial-targeting pre-sequence found in the gene for Arg II (25, 26). Co-Immunoprecipitation experiments and Western blots however demonstrated that Arg II is also found in crude SR preparations as well as immunoprecipitates of NOS1 (known to be found in the SR). Furthermore SR proteins (SERCA) were demonstrated in mitochondrial isolates and mitochondrial proteins in crude SR fractions. Although initially somewhat confusing it became apparent to us (and is consistent with the observations of others) that it remains virtually impossible to purify the mitochondria from the SR fraction and visa versa. This speaks to the tight spatial association and signal coupling between the mitochondria and machinery involved in excitation-contraction coupling (eg RYR channel). This interaction is critical because of the need for continuous regulation of the cellular oxidative energy generation in the mitochondria to the contractile work performed (For review see (51)). Thus our findings of Arg II expression in both mitochondria and SR fractions (most likely contaminated with mitochondrial membrane) is not inconsistent. Further it demonstrates that mitochondrial Arg II regulates concentrations of L-arginine in the microdomain of NOS1 thereby modulating RYR function.

### REFERENCES

1. Hare, J. M. (2004) Lancet 363, 1338-9.
2. Barouch, L. A., Harrison, R. W., Skaf, M. W., Rosas, G. O., Cappola, T. P., Kobeissi, Z. A., Hobai, I. A., Lemmon, C. A., Burnett, A. L., O'Rourke, B., Rodriguez, E. R., Huang, P. L., Lima, J. A., Berkowitz, D. E. & Hare, J. M. (2002) Nature 416, 337-9.
3. Khan, S. A., Skaf, M. W., Harrison, R. W., Lee, K., Minhas, K. M., Kumar, A., Fradley, M., Shoukas, A. A., Berkowitz, D. E. & Hare, J. M. (2003) Circ Res 92, 1322-9.
4. Khan, S. A., Lee, K., Minhas, K. M., Gonzalez, D. R., Raju, S. V., Tejani, A. D., Li, D., Berkowitz, D. E. & Hare, J. M. (2004) Proc Natl Acad Sci USA 101, 15944-8.
5. Hare, J. M. (2003) J Mol Cell Cardiol 35, 719-29.
6. Senzaki, H., Smith, C. J., Juang, G. J., Isoda, T., Mayer, S. P., Ohler, A., Paolocci, N., Tomaselli, G. F., Hare, J. M. & Kass, D. A. (2001) Faseb J 15, 1718-26.
7. Stamler, J. S., Lamas, S. & Fang, F. C. (2001) Cell 106, 675-83.
8. Hess, D. T., Matsumoto, A., Kim, S. O., Marshall, H. E. & Stamler, J. S. (2005) Nat Rev Mol Cell Biol 6, 150-66.
9. Champion, H. C., Skaf, M. W. & Hare, J. M. (2003) Heart Fail Rev 8, 35-46.
10. Berkowitz, D. E., White, R., Li, D., Minhas, K. M., Cemetich, A., Kim, S., Burke, S., Shoukas, A. A., Nyhan, D., Champion, H. C. & Hare, J. M. (2003) Circulation 108, 2000-6.
11. Cox, J. D., Kim, N. N., Traish, A. M. & Christianson, D. W. (1999) Nat Struct Biol 6, 1043-1047.
12. Morris, S. M., Jr., Kepka-Lenhart, D. & Chen, L. C. (1998) Am J Physiol 275, E740-7.
13. Demougeot, C., Prigent-Tessier, A., Marie, C. & Berthelot, A. (2005) J Hypertens 23, 971-8.
14. Johnson, F. K., Johnson, R. A., Peyton, K. J. & Durante, W. (2005) Am J Physiol Regul Integr Comp Physiol 288, R1057-62.
15. Ming, X. F., Barandier, C., Viswambharan, H., Kwak, B. R., Mach, F., Mazzolai, L., Hayoz, D., Ruffieux, J., Rusconi, S., Montani, J. P. & Yang, Z. (2004) Circulation 110, 3708-14.
16. Bivalacqua, T. J., Hellstrom, W. J., Kadowitz, P. J. & Champion, H. C. (2001) Biochem Biophys Res Commun 283, 923-7.
17. Meurs, H., Maarsingh, H. & Zaagsma, J. (2003) Trends Pharmacol Sci 24, 450-5.
18. Zimmermann, N., King, N. E., Laporte, J., Yang, M., Mishra, A., Pope, S. M., Muntel, E. E., Witte, D. P., Pegg, A. A., Foster, P. S., Hamid, Q. & Rothenberg, M. E. (2003) J Clin Invest 111, 1863-74.
19. Kocyigit, A., Zeyrek, D., Keles, H. & Koylu, A. (2004) Biol Trace Elem Res 102, 11-8.
20. Zhang, C., Hein, T. W., Wang, W., Chang, C. I. & Kuo, L. (2001) Faseb J 15, 1264-6.
21. Simon, A., Plies, L., Habermeier, A., Martine, U., Reining, M. & Closs, E. I. (2003) Circ Res 93, 813-20.
22. Closs, E. I., Scheld, J. S., Sharafi, M. & Forstermann, U. (2000) Mol Pharmacol 57, 68-74.
23. Brookes, P. S. (2004) Mitochondrion 3, 187-204.
24. Xu, K. Y., Huso, D. L., Dawson, T. M., Bredt, D. S. & Becker, L. C. (1999) Proc Natl Acad Sci USA 96, 657-62.
25. Shi, O., Kepka-Lenhart, D., Morris, S. M., Jr. & O'Brien, W. E. (1998) Mamm Genome 9, 822-4.
26. Gotoh, T., Sonoki, T., Nagasaki, A., Terada, K., Takiguchi, M. & Mori, M. (1996) FEBS Lett 395, 119-22.
27. Christianson, D. W. (2005) Acc Chem Res 38, 191-201.
28. Hare, J. M., Lofthouse, R. A., Juang, G. J., Colman, L., Ricker, K. M., Kim, B., Senzaki, H., Cao, S., Tunin, R. S. & Kass, D. A. (2000) Circ Res 86, 1085-92.
29. Gauthier, C., Leblais, V., Moniotte, S., Langin, D. & Balligand, J. L. (2000) Can J Physiol Pharmacol 78, 681-90.
30. Varghese, P., Harrison, R. W., Lofthouse, R. A., Georgakopoulos, D., Berkowitz, D. E. & Hare, J. M. (2000) J Clin Invest 106, 697-703.
31. Xu, L., Eu, J. P., Meissner, G. & Stamler, J. S. (1998) Science 279, 234-7.
32. Eu, J. P., Sun, J., Xu, L., Stamler, J. S. & Meissner, G. (2000) Cell 102, 499-509.
33. Eu, J. P., Xu, L., Stamler, J. S. & Meissner, G. (1999) Biochem Pharmacol 57, 1079-84.
34. Damy, T., Ratajczak, P., Shah, A. M., Camors, E., Marty, I., Hasenfuss, G., Marotte, F., Samuel, J. L. & Heymes, C. (2004) Lancet 363, 1365-7.
35. Haywood, G. A., Tsao, P. S., von der Leyen, H. E., Mann, M. J., Keeling, P. J., Trindade, P. T., Lewis, N. P., Byrne, C. D., Rickenbacher, P. R., Bishopric, N. H., Cooke, J. P., McKenna, W. J. & Fowler, M. B. (1996) Circulation 93, 1087-94.
36. Nathan, C. (1997) J Clin Invest 100, 2417-23.
37. Ziolo, M. T., Katoh, H. & Bers, D. M. (2001) Circulation 104, 2961-6.
38. Ziolo, M. T., Maier, L. S., Piacentino, V., 3rd, Bossuyt, J., Houser, S. R. & Bers, D. M. (2004) Circulation 109, 1886-91.
39. Moniotte, S., Kobzik, L., Feron, O., Trochu, J. N., Gauthier, C. & Balligand, J. L. (2001) Circulation 103, 1649-55.
40. Moniotte, S. & Balligand, J. L. (2002) Cardiovasc Drug Rev 20, 19-26.
41. Wang, W. W., Jenkinson, C. P., Griscavage, J. M., Kern, R. M., Arabolos, N. S., Byrns, R. E., Cederbaum, S. D. & Ignarro, L. J. (1995) Biochem Biophys Res Commun 210, 1009-16.
42. Sonoki, T., Nagasaki, A., Gotoh, T., Takiguchi, M., Takeya, M., Matsuzaki, H. & Mori, M. (1997) J Biol Chem 272, 3689-93.
43. Meurs, H., Hamer, M. A., Pethe, S., Vadon-Le Goff, S., Boucher, J. L. & Zaagsma, J. (2000) Br J Pharmacol 130, 1793-8.
44. Que, L. G., George, S. E., Gotoh, T., Mori, M. & Huang, Y. C. (2002) Nitric Oxide 6, 1-8.
45. Buga, G. M., Singh, R., Pervin, S., Rogers, N. E., Schmitz, D. A., Jenkinson, C. P., Cederbaum, S. D. & Ignarro, L. J. (1996) Am J Physiol 271, H1988-98.
46. Li, H., Meininger, C. J., Hawker, J. R., Jr., Haynes, T. E., Kepka-Lenhart, D., Mistry, S. K., Morris, S. M., Jr. & Wu, G. (2001) Am J Physiol Endocrinol Metab 280, E75-82.
47. Kaye, D. M., Pamell, M. M. & Ahlers, B. A. (2002) Circ Res 91, 1198-203.
48. Kaye, D. M., Ahlers, B. A., Autelitano, D. J. & Chin-Dusting, J. P. (2000) Circulation 102, 2707-12.
49. Levillain, O., Balvay, S. & Peyrol, S. (2005) J Histochem Cytochem 53, 533-41.
50. Levillain, O., Balvay, S. & Peyrol, S. (2005) Pflugers Arch 449, 491-503.
51. Csordas, G., Thomas, A. P. & Hajnoczky, G. (2001) Trends Cardiovasc Med 11, 269-75.
52. White, A. R., Ryoo, S., Li, D., Champion, H. C., Steppan, J., Wang, D., Nyhan, D., Shoukas, A. A., Hare, J. M. & Berkowitz, D. E. (2006) Hypertension In Press.

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating or preventing cardiac dysfunction in a subject comprising:
administering to the subject an effective amount of a compound that inhibits the expression or activity of Arginase II;
thereby treating or preventing cardiac dysfunction in a subject.

2. The method of claim 1, wherein the cardiac dysfunction is age related cardiac dysfunction.

3. A method of treating or preventing heart failure in a subject comprising:
administering to the subject an effective amount of a compound that inhibits the expression or activity of Arginase II;
thereby treating or preventing heart failure in a subject.

4. A method of treating or preventing vascular stiffness in a subject comprising:
administering to the subject an effective amount of a compound that inhibits the expression or activity of Arginase II;
thereby treating or preventing vascular stiffness in a subject.

5. A method of treating or preventing myocardial dysfunction in a subject by modulating the activity of Nitric Oxide Synthase 1 (NOS1) comprising:
contacting an Arginase II polypeptide, or a cell expressing an Arginase II polypeptide, with a compound that inhibits the expression or activity of Arginase II;
thereby modulating the activity of NOS1 and treating or preventing myocardial dysfunction in a subject.

6. The method of claim 1 wherein the compound inhibits the expression of Arginase II.

7. The method of claim 7, wherein the compound decreases the transcription or translation of Arginase II.

8. The method of claim 7, wherein the compound decreases the translation of Arginase II.

9. The method of claim 8, wherein the compound is a nucleic acid molecule.

10. The method of claim 9, wherein the nucleic acid molecule is an antisense RNA molecule, a siRNA molecule or a shRNA molecule.

11-15. (canceled)

16. A method of determining if a subject is at risk of developing heart failure or cardiac dysfunction comprising:
obtaining a biological sample from the subject;
determining the level of Arginase II in the sample;
wherein an elevated level Arginase II in the sample as compared to a control is indicative that the subject is at risk of developing heart failure or cardiac dysfunction.

17. The method of claim 16, wherein the cardiac dysfunction is age related cardiac dysfunction.

18. The method of claim 16, wherein the biological sample comprises cardiac myocytes.

19. The method of claim 16, wherein the level of Arginase II is determined by cellular imaging using a detectable antibody.

20. The method of claim 19, wherein the antibody is specific for Arginase II.

21. The method of claim 20, wherein the antibody is a monoclonal, polyclonal, humanized, human, or chimeric antibody, or a fragment thereof.

22. A method for treating or preventing age related cardiac dysfunction by modulating the activity of Arginase II comprising contacting the polypeptide or a cell expressing the polypeptide with a compound which binds to Arginase II in a sufficient concentration to modulate the activity of the to Arginase II.

23. A method for identifying a compound which modulates the activity of Arginase II comprising:
a) contacting Arginase II, or a cell expressing Arginase II with a test compound; and
b) determining whether the test compound binds to Arginase II.

24-30. (canceled)

31. A kit for the diagnosis of myocardial dysfunction or heart failure comprising an antibody specific for Arginase II, and instructions for use.

32. (canceled)
